(19) United States
(12) Patent Application Publication
(10) Pub. No.: US 2014/0171958 A1
(43) Pub. Date: Jun. 19, 2014

(54) Capture and Retrieval Device

(71) Applicant: Mirza Kamran Baig, Nottingham (GB)

(72) Inventor: Mirza Kamran Baig, Nottingham (GB)

(21) Appl. No.: 14/235,701

(22) PCT Filed: Jul. 27, 2012
(86) PCT No.: PCT/GB2012/051815

§ 371 Date: Jan. 30, 2014

(30) Foreign Application Priority Data
Jul. 29, 2011 (GB) 1113119.0

(51) Int. Cl. A61B 17/22

(52) U.S. Cl. CPC A61B 17/22032
USPC 606/108

(57) ABSTRACT

A device for capture and retrieval of a foreign body having a cavity, from a vessel of a patient, comprises a central shaft, radial balloon support means, radial inflatable balloon means and axial inflatable balloon means. The central shaft has a tip and an axial channel for receiving a guidewire therein. The radial balloon support means is attached to and extends from the central shaft and has a free end spaced therefrom. The radial inflatable balloon means is provided at said free end and is arranged to expand inwardly towards the central shaft upon inflation. The axial inflatable balloon means is provided on the central shaft, adjacent the tip thereof and is arranged to expand outwardly relative to the central shaft upon inflation.

This invention relates to a device for the capture and retrieval of foreign bodies, such as surgical tools and materials, from a vessel of a patient. Whilst the device of the present invention may be used for the capture and retrieval of substantially all kinds of foreign bodies from substantially all types of vessels, it has been developed particularly for the capture and retrieval of an embolised heart valve prosthesis from a vessel of a patient during a transcatheter aortic valve implantation (TAVI) procedure, and will therefore be described herein with particular emphasis on this application.

Transcatheter Aortic Valve Implantation (TAVI), also sometimes referred to as Percutaneous Aortic Valve Replacement (PAVR), is a recently developed surgical procedure, in which a heart valve prosthesis is transported to the heart carried on a balloon-tipped delivery catheter introduced into the patient's vessel, along a previously placed guidewire. The procedure is comparable to an intravascular angioplasty procedure performed when introducing a stent into a patient's vessel.

Aortic valve replacement previously required an invasive open heart surgery procedure, with considerable mortality and morbidity rates. Use of the TAVI procedure avoids the need for open heart surgery, thus significantly reducing both risk and cost.

A problem which can be encountered with this procedure is that the heart valve prosthesis can become prematurely detached from the delivery catheter (as is known also to occur with undeployed stents in the comparable intravascular angioplasty procedure), or may become dislodged from the correct anatomical delivery position even after initial successful deployment, and can thus become embolised within the patient's vessel. An embolised heart valve prosthesis necessitates immediate retrieval in order to avoid exposing the patient to severe risk. This may require immediate invasive open heart surgery, thus negating many of the benefits of the TAVI procedure.

A device for the retrieval of an undeployed stent during an intravascular angioplasty procedure is described in the applicant's co-pending European Patent Application published as EP 1,587,447. However, in view of the physical differences between a stent and a heart valve prosthesis, such a device would not be suitable for retrieval of a heart valve prosthesis during a TAVI procedure. The present invention builds on the general principles of the device described in EP 1,587,447 to provide a modified retrieval device adapted so as to be particularly suitable for the capture and retrieval of a heart valve prosthesis during a TAVI procedure, without the need for invasive surgery.

According to the present invention, there is provided a device for capture and retrieval of a foreign body having a cavity, from a vessel of a patient, which device comprises:
a central shaft having a tip, and an axial channel for receiving a guidewire therein;
radial balloon support means attached to and extending from the central shaft and having a free end spaced therefrom;
radial inflatable balloon means provided at said free end and arranged to expand inwardly towards the central shaft upon inflation; and
axial inflatable balloon means provided on the central shaft, adjacent the tip thereof and arranged to expand outwardly relative to the central shaft upon inflation;

whereby in use the device is positioned such that the tip of the central shaft is inserted into, and passes through, a cavity of the foreign body to be retrieved, such that subsequent inflation of the axial inflatable balloon means serves to prevent removal of the foreign body from the device, whilst subsequent inflation of the radial inflatable balloon means causes said radial inflatable balloon means to bear against an external surface of the foreign body.

In preferred embodiments of the present invention, the device is adapted for capture and retrieval of an embolised heart valve prosthesis, in particular a stented heart valve prosthesis (sometimes referred to as a heart valve stent), from a vessel of a patient, in particular the aorta or left ventricle. In use the device is thus positioned such that the tip of the central shaft is inserted into, and passes through, the central cavity of the prosthesis to be captured, such that subsequent inflation of the axial inflatable balloon means ensures the prosthesis is retained on the device, and prevents re-embolisation of the prosthesis. Subsequent inflation of the radial inflatable balloon means then causes said radial inflatable balloon means to bear against an external surface of the prosthesis to grasp and/or compress it against the central shaft.

The radial inflatable balloon means is preferably arranged so as in use to bear against the prosthesis at two or more spaced locations around the circumference thereof. More preferably, the radial inflatable balloon means is generally annular, and is arranged such that the central shaft passes through the centre thereof.

The radial balloon support means preferably is or includes a generally cylindrical sleeve spaced radially from and surrounding said central shaft, and extending axially relative thereto, to define a generally cylindrical recess between the sleeve and the central shaft.

In addition to its role in grasping the external surface of the prosthesis, the radial inflatable balloon means may also subsequently be utilised to compress the captured prosthesis against the central shaft, to ensure that the prosthesis remains engaged with the device during withdrawal of the device from the vessel. Deflation of the axial inflatable balloon means may be required in order to facilitate this.

In addition to its role in preventing re-embolisation of the captured prosthesis, the axial inflatable balloon means also serves to align the prosthesis with the central shaft prior to grasping of the prosthesis by the radial inflatable balloon means.

To enable the radial and axial inflatable balloon means to fulfil these additional functions, the device is preferably constructed such that the tip of the central shaft extends beyond the free end of the radial balloon support means. More preferably, the radial and axial inflatable balloon means are offset relative to one another, such that the axial inflatable balloon means presents to, and passes through, the prosthesis cavity prior to the radial inflatable balloon means presenting to the external surface of the prosthesis.

In an alternative embodiment of the present invention, the radial balloon support means is detachable from the central shaft, and is adapted to be withdrawn from the vessel independently of the central shaft following capture and compression of the prosthesis, and subsequent deflation of the radial inflatable balloon means.

In order that the present invention may be fully understood, preferred embodiments thereof will now be described in detail, though only by way of example, with reference to the accompanying drawings, in which:

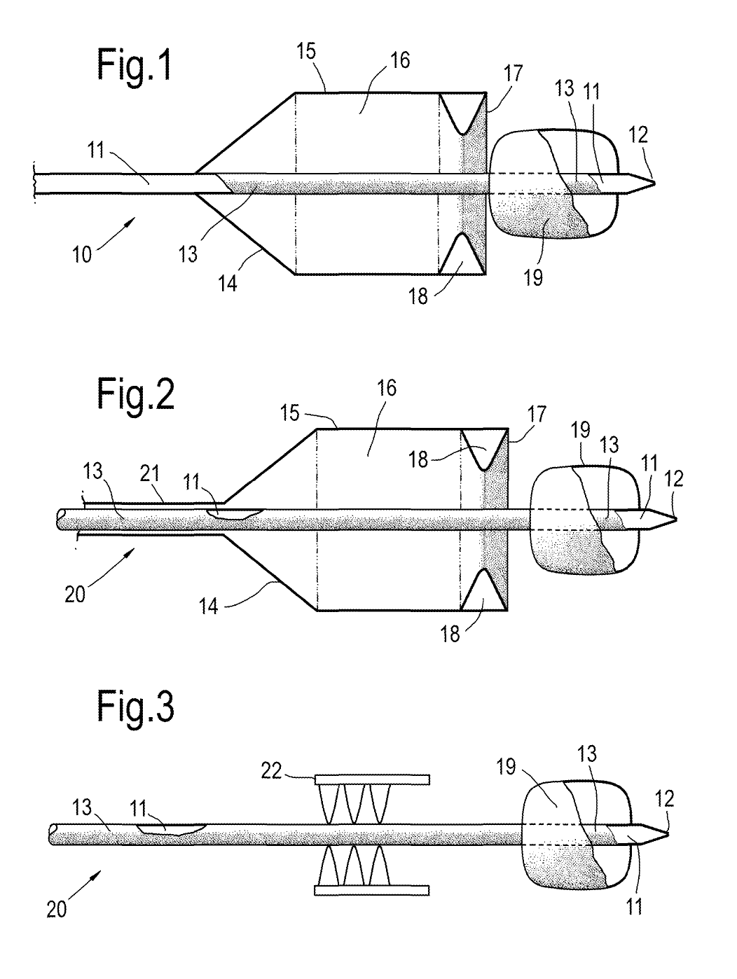

FIG. 1 shows a cross-sectional side view of a first embodiment of a capture and retrieval device according to the present invention;

FIG. 2 shows a cross-sectional side view of a second embodiment of a capture and retrieval device according to the present invention; and

FIG. 3 shows a cross-sectional side view of the device of Figure 2, following capture of a prosthesis and subsequent withdrawal of the radial balloon support means.

Referring first to Figure 1, there is shown a first embodiment of capture and retrieval device, generally indicated 10, adapted for capture and retrieval of an embolised stented heart valve prosthesis from a vessel of a patient. The device 10 comprises a central shaft 11 having a tip 12, with an axial channel 13 for receiving a guidewire running through the shaft 11. Radial balloon support means 14, including a generally cylindrical sleeve portion 15, attach to and extend from the central shaft 11. The generally cylindrical sleeve portion 15 is spaced radially from and surrounds the central shaft 11, and extends axially relative thereto, to define a generally cylindrical recess 16 between the sleeve and the central shaft 11.

The radial balloon support means 14 has a free end 17 at which is provided generally annular radial inflatable balloon means 18, arranged such that the central shaft 11 passes through the centre thereof. The radial inflatable balloon means 18 is arranged to expand inwardly towards the central shaft 11 upon inflation. Axial inflatable balloon means 19 are provided on the central shaft 11, adjacent the tip 12, and are arranged to expand outwardly relative to the shaft 11 upon inflation.

As can be seen from Figure 1, the tip 12 of the central shaft 11 extends beyond the free end 17 of the radial balloon support means 14. As can also be seen, the radial and axial balloon means 18, 19 are offset relative to one another, such that the axial balloon means 19 present to the target prosthesis prior to the radial balloon means 18.

In use, for the capture and retrieval of an embolised stented heart valve prosthesis from a patient's vessel, the device 10 is introduced into the patient's vessel via a delivery catheter and guidewire, which will already be in place from the TAVI procedure during which the prosthesis became embolised. With the radial and axial balloon means 18, 19 deflated, the tip 12 of the device 10 is inserted into, and passes through, the central cavity of the prosthesis, such that the prosthesis is received within the generally cylindrical recess 16. The axial balloon means 19 is then inflated in order partially to capture the prosthesis within the recess 16, and to align the prosthesis with the central shaft 11 of the device 10. Alignment of the prosthesis with the shaft 11 is particularly required where the embolised prosthesis is disposed at an angle relative to the axis of the central shaft 11.

Once the prosthesis is correctly aligned, the radial balloon means 18 is then inflated to bear against the external surface of the prosthesis. The prosthesis is thus firmly grasped (captured) ready for retrieval.

If desired, the radial balloon means 18 can be inflated further in order to compress the captured prosthesis against the central shaft 11. This serves to ease retrieval of the combined device 10 and prosthesis ensemble, and to ensure the prosthesis remains engaged with the device 10 during retrieval. Deflation of the axial balloon means 19 may be required during this process.

The combined device 10 and prosthesis ensemble is then withdrawn through the delivery catheter. Deflation of the radial 18 and/or axial balloon means 19 may be required during some or all of this process to facilitate retrieval of the combined device 10 and prosthesis ensemble. Alternatively, the combined device 10, prosthesis and delivery catheter may be withdrawn as an ensemble, leaving the guidewire in situ for further procedures.

Referring now to Figure 2, there is shown a second embodiment of capture and retrieval device, generally indicated 20, adapted for capture and retrieval of an embolised stented heart valve prosthesis from a vessel of a patient. The second embodiment 20 is similar in many respects to the first embodiment 10 described above, and like reference numerals are used herein where appropriate. The second embodiment 20 differs from the first embodiment 10 in that the radial balloon support means 14 have a detachable connection 21 with the central shaft 11.

In use, for the capture and retrieval of an embolised stented heart valve prosthesis from a patient's vessel, the device 20 is deployed in the same manner as described above with reference to the first embodiment 10, up to and including the capture of the embolised prosthesis. The captured prosthesis is then compressed against the central shaft 11 by inflation of the radial balloon means 18 as also described above with reference to the first embodiment 10. Once the prosthesis has been compressed, the radial balloon means 18 are deflated, and the detachable connection 21 between the radial balloon support means 14 and the central shaft 11 activated, such that the radial balloon support means 14 are then withdrawn through the delivery catheter independently from the remainder of the device 20.

Referring now to Figure 3, this leaves the prosthesis 22 compressed against the central shaft 11, and renders retrieval of the combined device 20 and prosthesis 22 ensemble through the delivery catheter considerable easier, due to the absence of the radial balloon support means 14. The axial balloon means 19 remain and can be re-inflated if necessary to ensure the prosthesis 22 remains engaged on the device 20 during withdrawal. The combined device 20 and prosthesis 22 ensemble may either be withdrawn through the delivery catheter, or alternatively, the combined device 20, prosthesis 22 and delivery catheter may be withdrawn as an ensemble, leaving the guidewire in situ for further procedures, as with the first embodiment 10.

1. A capture and retrieval device for capture and retrieval of a foreign body having a cavity, from a vessel of a patient, which device comprises:
a central shaft having a tip, and an axial channel for receiving a guidewire therein;
a radial balloon support attached to and extending from the central shaft and having a free end spaced therefrom;
a radial inflatable balloon provided at the free end and arranged to expand inwardly towards the central shaft upon inflation; and
an axial inflatable balloon provided on the central shaft, adjacent the tip thereof and arranged to expand outwardly relative to the central shaft upon inflation;
whereby in use the capture and retrieval device is positioned such that the tip of the central shaft is inserted into, and passes through, a cavity of said foreign body to be retrieved, such that subsequent inflation of the axial inflatable balloon serves to prevent removal of said foreign body from the device, whilst subsequent inflation of the radial inflatable balloon causes the radial inflatable balloon to bear against an external surface of said foreign body.

2. The capture and retrieval device of claim 1, wherein said foreign body to be retrieved is a heart valve prosthesis.

3. The capture and retrieval device of claim 2, wherein said heart valve prosthesis is a stented heart valve prosthesis.

4. The capture and retrieval device of claim 2, wherein the radial inflatable balloon is arranged so as in use to bear against said prosthesis at at least two spaced locations around said prosthesis's circumference.

5. The capture and retrieval device of claim 1, wherein the tip of the central shaft extends beyond the free end of the radial balloon support.

6. the capture and retrieval device of claim 5, wherein the radial inflatable balloon and the axial inflatable balloon are offset relative to one another.

7. The capture and retrieval device of claim 1, wherein the radial inflatable balloon is generally annular, and is arranged such that the central shaft passes through the annular radial inflatable balloon's centre.

8. The capture and retrieval device of claim 1, wherein the radial balloon support comprises a generally cylindrical sleeve spaced radially from and surrounding the central shaft, and extending axially relative thereto, thereby to define a generally cylindrical recess between said sleeve and the central shaft.

9. The capture and retrieval device of claim 1, wherein the radial balloon support is detachable from the central shaft, and is adapted to be withdrawn from said vessel independently of the central shaft following capture of said foreign body, and subsequent deflation of the radial inflatable balloon.
